SEQUENCE LISTING (10 sequences)

SEQ ID NO: 1 - DNA, 399 bp, Artificial Sequence
  Nucleic acid sequence corresponding to the first DNA fragment of the vehicle fragment
atggccaaactcgaaaccgttacgctgggcaacatcggcaaggatggcaagcagacgctggtgctgaatccgcgcggtgttaatccgaccaatggtgtggcgagtctgagtcaagcgggtgccgttccagcgctggagaaacgtgtgaccgttagtgtgagccagccgagtcgcaaccgcaagaactacaaggtgcaagtgaagattcagaacccaaccgcgtgcaccgccaacggtagttgcgatccaagcgtgacccgccaagcgtacgccgatgttaccttcagcttcacccagtacagcaccgacgaagagcgcgcgtttgttcgcaccgaactggccgcgctgctggcgagtccactgctgatcgatgcgattgaccagctgaacccagcctac

SEQ ID NO: 2 - DNA, 39 bp, Artificial Sequence
  Nucleic acid sequence corresponding to the second DNA fragment of the capture fragment according to the first embodiment
gattgcgcgtggcatctgggcgaactggtgtggtgcacc

SEQ ID NO: 3 - protein, 133 aa, Artificial Sequence
  Amino acid sequence of the vehicle fragment
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 4 - protein, 13 aa, Artificial Sequence
  Amino acid sequence of the capture fragment according to the first embodiment
DCAWHLGELVWCT

SEQ ID NO: 5 - DNA, 447 bp, Artificial Sequence
  Nucleic acid sequence corresponding to the DNA fragment of the fusion peptide according to the first embodiment
atggccaaactcgaaaccgttacgctgggcaacatcggcaaggatggcaagcagacgctggtgctgaatccgcgcggtgttaatccgaccaatggtgtggcgagtctgagtcaagcgggtgccgttccagcgctggagaaacgtgtgaccgttagtgtgagccagccgagtcgcaaccgcaagaactacaaggtgcaagtgaagattcagaacccaaccgcgtgcaccgccaacggtagttgcgatccaagcgtgacccgccaagcgtacgccgatgttaccttcagcttcacccagtacagcaccgacgaagagcgcgcgtttgttcgcaccgaactggccgcgctgctggcgagtccactgctgatcgatgcgattgaccagctgaacccagcctacggctctggcgattgcgcgtggcatctgggcgaactggtgtggtgcacc

SEQ ID NO: 6 - protein, 149 aa, Artificial Sequence
  Amino acid sequence of the fusion peptide according to the first embodiment
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYGSGDCAWHLGELVWCT

SEQ ID NO: 7 - DNA, 294 bp, Artificial Sequence
  Nucleic acid sequence corresponding to the second DNA fragment of the capture fragment according to the second embodiment
gcgatggtggacaacaagttcaacaaggagcagcagaacgcgttttacgagattctgcatctgccaaatctgaatgaagaacagcgcaacgcgttcattcaaagtctgaaagacgacccgagtcagagtgccaatctgctggccgaagccaaaaaactgaacgatgcgcaagccccgaaaaatctgaacgaggagcagcgtaacgcctttatccagagtctgaaggacgacccgagccagagcgccaatctgctggcggaagccaaaaagctgaatgacgcccaagccccgaaa

SEQ ID NO: 8 - protein, 98 aa, Artificial Sequence
  Amino acid sequence of the capture fragment according to the second embodiment
AMVDNKFNKEQQNAFYEILHLPNLNEEQRNAFIQSLKDDPSQSANLLAEAKKLNDAQAPKNLNEEQRNAFIQSLKDDPSQSANLLAEAKKLNDAQAPK

SEQ ID NO: 9 - DNA, 720 bp, Artificial Sequence
  Nucleic acid sequence corresponding to the DNA fragment of the fusion peptide according to the second embodiment
atggccaaactcgaaaccgttacgctgggcaacatcggcaaggatggcaagcagacgctggtgctgaatccgcgcggtgttaatccgaccaatggtgtggcgagtctgagtcaagcgggtgccgttccagcgctggagaaacgtgtgaccgttagtgtgagccagccgagtcgcaaccgcaagaactacaaggtgcaagtgaagattcagaacccaaccgcgtgcaccgccaacggtagttgcgatccaagcgtgacccgccaagcgtacgccgatgttaccttcagcttcacccagtacagcaccgacgaagagcgcgcgtttgttcgcaccgaactggccgcgctgctggcgagtccactgctgatcgatgcgattgaccagctgaacccagcctacggcggtgcgagtgaaagtggcgcggcgatggtggacaacaagttcaacaaggagcagcagaacgcgttttacgagattctgcatctgccaaatctgaatgaagaacagcgcaacgcgttcattcaaagtctgaaagacgacccgagtcagagtgccaatctgctggccgaagccaaaaaactgaacgatgcgcaagccccgaaaaatctgaacgaggagcagcgtaacgcctttatccagagtctgaaggacgacccgagccagagcgccaatctgctggcggaagccaaaaagctgaatgacgcccaagccccgaaataa

SEQ ID NO: 10 - protein, 239 aa, Artificial Sequence
  Amino acid sequence of the fusion peptide according to the second embodiment
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYGGASESGAAMVDNKFNKEQQNAFYEILHLPNLNEEQRNAFIQSLKDDPSQSANLLAEAKKLNDAQAPKNLNEEQRNAFIQSLKDDPSQSANLLAEAKKLNDAQAPK

(19) United States
(12) Patent Application Publication
(10) Pub. No.: US 2022/0098236 A1
(43) Pub. Date: Mar. 31, 2022

(54) Fusion Peptide for Forming Virus-Like Particle

(71) Applicant: NATIONAL SUN YAT-SEN UNIVERSITY, Kaohsiung City (TW)

(72) Inventors: Hong-Wei Yang, Kaohsiung City (TW); Hao-Han Pang, Kaohsiung City (TW); Ying-Pei Hsu, Kaohsiung City (TW); Nan-Si Li, Kaohsiung City (TW)

(21) Appl. No.: 17/485,680

(22) Filed: Sep. 27, 2021

Related U.S. Application Data
(60) Provisional application No. 63/083,872, filed on Sep. 26, 2020.

(51) Int. Cl. C07K 2/00

(52) U.S. Cl. CPC C07K 2/00; C07K 2319/30

(57) ABSTRACT

A fusion peptide for forming a virus-like particle is used to solve the problem that a target biomolecule is chemically conjugated to the virus-like particle. The fusion peptide includes a vehicle fragment and a capture fragment connected to the vehicle fragment. The vehicle fragment is for forming the virus-like particle, and the capture fragment is for forming a capture peptide which is not enveloped by the virus-like particle and is adapted to specific bind the target biomolecule.

### CROSS REFERENCE TO RELATED APPLICATIONS

The application claims the benefit of U.S. provisional application No. 63/083,872, filed on Sep. 26, 2020, and the entire contents of which are incorporated herein by reference.

### BACKGROUND OF THE INVENTION

### 1. Field of the Invention

The present invention generally relates to a fusion peptide and, more particularly, to a fusion peptide for forming a virus-like particle.

### 2. Description of the Related Art

Virus-like particle (VLP) with great biocompatibility and biosafety is considered as a potential delivery vehicle or a potential therapeutic vehicle. As an example, the therapeutic agent (such as chemotherapy drugs) can be enveloped in the virus-like particle (VLP) and then delivered to its target site.

In order to target the specific target site, it is known to chemical conjugated a biomolecule (such as an antibody) to the virus-like particle (VLP). The virus-like particle (VLP) carrying the antibody can thus target the specific target site (such as the target antigen that can specifically bind to the antibody or a cell includes the target antigen) via the interaction between the antibody and the target antigen. However, a crosslinker is needed for performing the chemical conjugation reaction, and the chemical conjugation reaction needs a longer time period, affecting the stability of the biomolecule (such as the antibody).

In light of this, it is necessary to provide a fusion peptide for forming the virus-like particle.

### SUMMARY OF THE INVENTION

It is therefore an objective of the present invention to provide a fusion peptide for forming a virus-like particle, which is able to bind to a target biomolecule.

It is another objective of the present invention to provide a fusion peptide for forming a virus-like particle, which is able to bind to the target biomolecule without any crosslinker.

One embodiment of the present invention discloses a fusion peptide for forming virus-like particle. The fusion peptide can include a vehicle fragment and a capture fragment connected to the vehicle fragment. The vehicle fragment is used to form a virus-like particle. The capture fragment is used to form a capture peptide that is not enveloped by the virus-like particle and is able to specifically bind to a target biomolecule.

Accordingly, instead of enveloping in the virus-like particle formed by the vehicle fragment, the fusion peptide for forming virus-like particle according to the present invention has the capture peptide formed by the capture fragment that is exposed outside the virus-like particle. Therefore, the capture peptide can specifically bind to a target biomolecule to form the virus-like particle with the target biomolecule, and the target biomolecule can be delivered to a predetermined location by the virus-like particle. Alternatively, by the specific binding between the capture peptide and the target biomolecule, the targeting of the virus-like particle can also be improved.

Moreover, the virus-like particle carrying the target biomolecule is formed by a reversible specific binding (such as the interaction of hydrogen bond, van der Waals force, electrostatic force, hydrophobic interaction, etc.) between the capture peptide and the target biomolecule. Therefore, the process for conjugating the target biomolecule to the virus-like particle (VLP) using the crosslinker can be omitted. As such, the virus-like particle (VLP) carrying the target biomolecule can be manufactured at a decreased cost, and the virus-like particle (VLP) carrying the target biomolecule can be manufactured with a better yield.

In a preferred form shown, the fusion peptide can further include a linker fragment connected between the vehicle fragment and the capture fragment. As such, the folding of the vehicle fragment and the folding of the capture fragment will not affect each other; and thus, the biological activity of the capture peptide formed by the capture fragment can be remained.

In a preferred form shown, the vehicle fragment can has an amino acid sequence set forth as SEQ ID NO: 3, and the capture fragment can has an amino acid sequence set forth as SEQ ID NO: 4. Preferably, the fusion peptide can consist of an amino acid sequence set forth as SEQ ID NO: 6. As such, the capture peptide with an IgG Fc-binding domain that is able to bind to a Fc domain of an antibody (such as the Fc domain of an anti-RBD antibody); and thus, the fusion peptide can form the virus-like particle (VLP) carrying the antibody by the specific binding between the IgG Fc-binding domain and the Fc domain. The antibody carried by the virus-like particle (VLP) can specifically bind to a virus particle (such as such as specifically bind to the virus particle of SARS-CoV-2 via the receptor-binding domain of SARS-CoV-2). As a result, the specific binding between the virus particle and the receptor of the target cell (such as angiotensin-converting enzyme 2 (ACE2)) can be blocked, and the infection of the target cell by the virus particle can be prevented.

In a preferred form shown, the vehicle fragment can has an amino acid sequence set forth as SEQ ID NO: 3, and the capture fragment can has an amino acid sequence set forth as SEQ ID NO: 8. Preferably, the fusion peptide can consist of an amino acid sequence set forth as SEQ ID NO: 10. As such, the capture peptide with a z domain that is able to bind to a Fc domain of an antibody (such as the Fc domain of an anti-RBD antibody); and thus, the fusion peptide can form the virus-like particle (VLP) carrying the antibody by the specific binding between the z domain and the Fc domain. The antibody carried by the virus-like particle (VLP) can specifically bind to a virus particle (such as such as specifically bind to the virus particle of SARS-CoV-2 via the receptor-binding domain of SARS-CoV-2). As a result, the specific binding between the virus particle and the receptor of the target cell (such as angiotensin-converting enzyme 2 (ACE2)) can be blocked, and the infection of the target cell by the virus particle can be prevented.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts a transmission electron microscopy (TEM) image of the virus-like particle (VLP) formed by the fusion peptide according to the first embodiment of the present invention in trial (A).

FIG. 2 depicts a transmission electron microscopy (TEM) image of the virus-like particle (VLP) formed by the fusion peptide according to the second embodiment of the present invention in trial (A).

FIG. 3 depicts a Western blot image of the virus-like particle (VLP) formed by the fusion peptide according to the first embodiment of the present invention (group B1) and the virus-like particle (VLP) formed by the vehicle fragment (group B2) in trial (B). Lane “L” is pre-stained protein marker. The band labeled with D indicates the band of dimer of the fusion peptide, while the band labeled with M indicates the band of monomer of the fusion peptide.

FIG. 4 depicts a Western blot image of the virus-like particle (VLP) formed by the vehicle fragment (group C1) and the virus-like particle (VLP) formed by the fusion peptide according to the second embodiment of the present invention (group C2) in trial (C). Lane “L” is pre-stained protein marker. The band labeled with D indicates the band of dimer of the fusion peptide, while the band labeled with M indicates the band of monomer of the fusion peptide.

FIG. 5 depicts a bar chart illustrating the absorbance of the complex including the virus-like particle (VLP) formed by the fusion peptide according to the first embodiment of the present invention of groups D1 to D6 at 450 nm in trial (D). “*” indicates significant difference compared to group D1 (p<0.05).

FIG. 6 depicts an image of agarose gel electrophoresis of the mixture including the virus-like particle (VLP) formed by the fusion peptide according to the second embodiment of the present invention and the primary antibody of groups E1 to E7 in trial (E).

FIG. 7 depicts a bar chart illustrating the binding strength between the mixture of groups H1 to H3 and angiotensin-converting enzyme 2 (ACE2) immobilized on the well in trial (H). “*” indicates significant difference compared to group H1 (p<0.05).

FIG. 8 depicts a schematic diagram illustrating the relative position of the first sprayer S1, the second sprayer S2, the upper chip C1 and the lower chip C2 in the sealed glass box in trail (I).

FIG. 9 depicts a bar chart illustrating the absorbance of the upper chip C1, as well as the lower chip C2, of groups I1 to 12 at 450 nm in trial (I). “*” indicates significant difference compared to group I1 (p<0.05).

FIG. 10 depicts a schematic diagram illustrating the relative position of the first sprayer S1 and the second sprayer S2 in the sealed glass box in trial (J).

FIG. 11 depicts a schematic diagram illustrating the blue laser beam B passing through the sealed glass box in trial (J).

FIG. 12 depicts the optical path formed by the blue laser beam B at 0 minute in the situation that only the SARS-CoV-2 mimicking signal source is suspended in the sealed glass box (group J1) in trial (J).

FIG. 13 depicts the optical path formed by the blue laser beam B at 40 minutes in the situation that only the SARS-CoV-2 mimicking signal source is suspended in the sealed glass box (group J1) in trial (J).

FIG. 14 depicts the optical path formed by the blue laser beam B at 0 minute in the situation that both the SARS-CoV-2 mimicking signal source and the virus-like particle (VLP) formed by the fusion peptide according to the second embodiment of the present invention carrying the anti-RBD antibody are suspended in the sealed glass box (group J2) in trial (J).

FIG. 15 depicts the optical path formed by the blue laser beam B at 40 minutes in the situation that both the SARS-CoV-2 mimicking signal source and the virus-like particle (VLP) formed by the fusion peptide according to the second embodiment of the present invention carrying the anti-RBD antibody are suspended in the sealed glass box (group J2) in trial (J).

### DETAILED DESCRIPTION OF THE INVENTION

Herein, the term “fusion peptide” indicates a peptide manufactured by recombinant DNA technology. The peptide includes at least two amino acid fragments respectively encoded by corresponding genes that originally coded for separate portions, which can be appreciated by a person having ordinary skill in the art; and therefore, detail description is not given to avoid redundancy.

Specifically, the fusion peptide for forming a virus-like particle (VLP) according to the present invention can include a vehicle fragment and a capture fragment connected to the vehicle fragment. The vehicle fragment is used for forming the virus-like particle (VLP), while the capture fragment is used for forming a capture peptide that is not enveloped by and is exposed outside the virus-like particle (VLP). The capture peptide can specifically bind to a target biomolecule. As an example, the target biomolecule can be an antibody, an antigen, an enzyme, a substrate, an aptamer or a ligand.

The fusion peptide can be expressed by E. coli cells. As an example, an expression plasmid for expressing the fusion peptide can be constructed and transformed into the E. coli cells. The fusion peptide expressed by the E. coli cells can be purified, and the purified fusion peptide can therefore be obtained.

Specifically, the expression plasmid includes a first DNA fragment corresponding to the vehicle fragment and a second DNA fragment corresponding to the capture fragment. Moreover, the first and second DNA fragments preferably have the codon usage of E. coli, thus the E. coli cells can show preferable expression efficiency. In the first embodiment of the present invention, the first and second DNA fragments have nucleic acid sequences set forth as SEQ ID NOS: 1 and 2, respectively. In addition, the vehicle fragment and the capture fragment expressed by the E. coli cells have the amino acid sequences set forth as SEQ ID NOS: 3 and 4, respectively.

Furthermore, a linker fragment can be provided to connect between the vehicle fragment and the capture fragment. The sequence of the linker fragment can be appreciated by a person having ordinary skill in the art; and therefore, detail description is not given to avoid redundancy. In the first embodiment of the present invention, the expression plasmid comprises a nucleic acid sequence set forth as SEQ ID NO: 5, while the fusion peptide expressed by the E. coli cells consists of an amino acid sequence set forth as SEQ ID NO: 6.

The construction of the expression plasmid is the ordinary skill in the art, and therefore is not limited to the following statement. In the first embodiment of the present invention, the DNA fragment with the nucleic acid sequence set forth as SEQ ID NO: 5 is synthesized and digested by the restriction enzyme. The digested DNA fragment is then ligated to a pCDFDuet-1 vector by a ligase, and the expression plasmid for expressing the fusion peptide according to the first embodiment is obtained.

Subsequently, after the expression plasmid is transformed into the E. coli BL21(DE) cells, the E. coli BL21(DE) cells can express the fusion peptide by isopropyl β-D-1-thiogalactopyranoside (IPTG) induction. The fusion peptide expressed by the E. coli BL21(DE) cells is obtained and is precipitated by ammonium sulfate ((NH₄)₂SO₄) to obtain a crude sample. The crude sample is then resuspended in phosphate buffered saline (PBS) and is extracted by a mixture including n-butanol and chloroform in a volume ratio of 1:1. A upper aqueous layer solution is collected and is purified by sucrose gradient ultracentrifugation. After precipitating by a salt solution including polyethylene glycol 8000 (20% w:v PEG 8000-NaCl solution), the obtained precipitate is resuspended in phosphate buffered saline (PBS). The purified fusion peptide can be obtained after dialysis with phosphate buffered saline (PBS).

The purified fusion peptide has the capture peptide with an immunoglobulin G (IgG) fragment-crystllizable (Fc)-binding domain that is able to bind to a fragment-crystllizable (Fc) domain of an antibody; and thus, the fusion peptide can be used as a vehicle of the antibody for specific binding to an antigen of a virus particle. As a result, the specific binding between the virus particle and a receptor of a target cell can be blocked, and the infection of the target cell by the virus particle can be prevented. As an example, in order to prevent the target cell from infection by severe acute respiratory syndrome coronavirus 2 (SARS-CoV-2), the antibody can be an anti-RBD antibody that is able to specific bind to the receptor-binding domain (RBD domain) of SARS-CoV-2. By the specific binding between the anti-RBD antibody and the RBD domain of SARS-CoV-2, the specific binding between the RBD domain of SARS-CoV-2 and angiotensin-converting enzyme 2 (ACE2) of the target cell can be blocked.

Based on the same technical concept, in the second embodiment of the present invention, the first and second DNA fragments have nucleic acid sequences set forth as SEQ ID NOS: 1 and 7, respectively. In addition, the vehicle fragment and the capture fragment expressed by the E. coli cells have the amino acid sequences set forth as SEQ ID NOS: 3 and 8, respectively. The expression plasmid comprises a nucleic acid sequence set forth as SEQ ID NO: 9, while the fusion peptide expressed by the E. coli cells consists of an amino acid sequence set forth as SEQ ID NO: 10.

Moreover, after the construction of the expression plasmid for expressing the fusion peptide according to the second embodiment (the vector that used for the expression plasmid is pCDFDuet-1 vector), the purified fusion peptide can be obtained according to the same procedure. The purified fusion peptide has the capture peptide with a z domain that is able to bind to the Fc domain of the antibody; and thus, the fusion peptide can be used as the vehicle of the antibody for specific binding to the antigen of the virus particle. As a result, the specific binding between the virus particle and the receptor of the target cell can be blocked, and the infection of the target cell by the virus particle can be prevented.

To evaluate both the fusion peptides according to the first and second embodiments of the present invention form the virus-like particles (VLPs), and both the capture fragments formed by the capture peptides are not enveloped in the virus-like particles (VLPs) and are able to specifically bind to the target biomolecule, the following trials are carried out.

Trial (A).

In trial (A), the fusion peptide according to the first embodiment of the present invention (5 μL) is pipetted onto the Formvar-coated copper mesh grids for 5 minutes, followed by exposure to the aqueous uranyl acetate solution (8 μL) for 2 minutes as a negative stain. Excess stain is then removed, and the Formvar-coated copper mesh grids are left to dry in air overnight. The obtained sample is analyzed by transmission electron microscopy (TEM) (75 keV accelerating voltage). Moreover, the fusion peptide according to the second embodiment of the present invention is analyzed according to the same procedure.

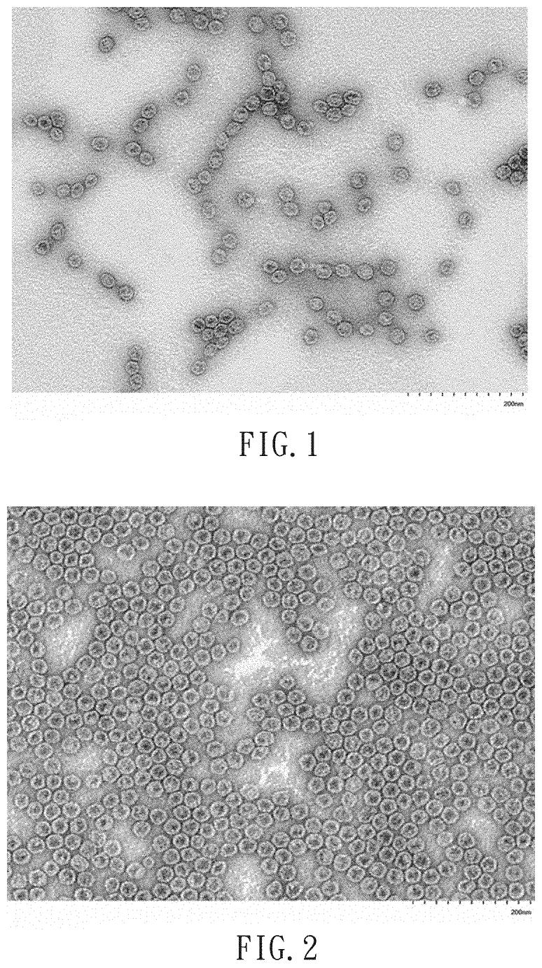

Referring to FIGS. 1 and 2, both the virus-like particles (VLPs) formed by the fusion peptide according to the first and second embodiments of the present invention form contact morphology.

Trial (B).

In trial (B), the virus-like particle (VLP) formed by the fusion peptide according to the first embodiment of the present invention (group B1) and the virus-like particle (VLP) formed by the vehicle fragment (group B2) are analyzed by sodium dodecyl sulfate polyacrylamide gel electrophoresis (SDS-PAGE, 15%). The proteins on PAGE are transferred to a poly(vinylidene) fluoride (PVDF) membrane. The poly(vinylidene) fluoride (PVDF) membrane is then blocked using a milk solution (5%). The poly(vinylidene) fluoride (PVDF) membrane is then performed antibody binding using a rabbit anti-mouse IgG primary antibody. After tris(hydroxymethyl)aminomethane (Tris) buffer (TBST) with polysorbate 20 (Tween 20) washing for three times, the poly(vinylidene) fluoride (PVDF) membrane is stained using a horseradish peroxidase (HRP)-labelled goat anti-rabbit IgG secondary antibody. Finally, after washing for three times, the poly(vinylidene) fluoride (PVDF) membrane is photographed after soaking in a Western luminal substrate.

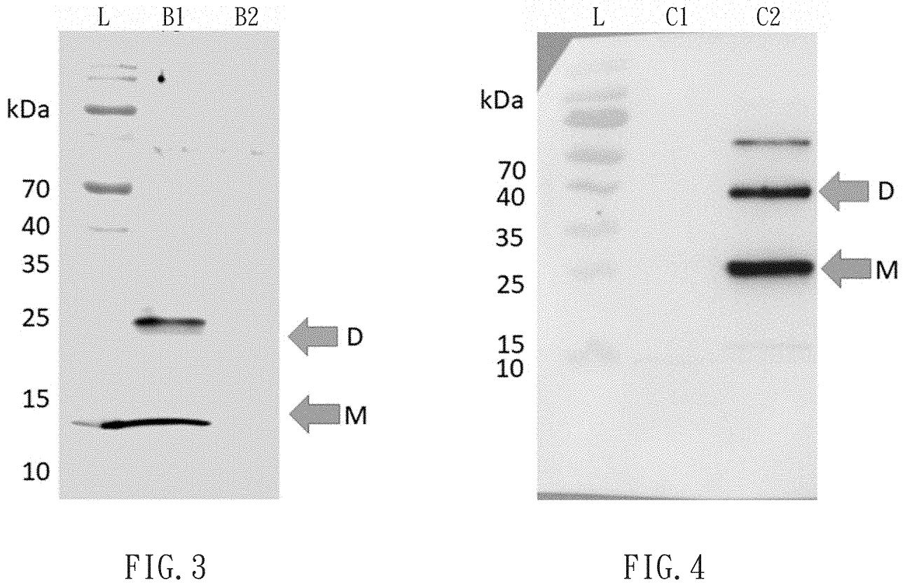

Referring to FIG. 3, the horseradish peroxidase (HRP)-labelled goat anti-rabbit IgG secondary antibody can only recognize the virus-like particle (VLP) formed by the fusion peptide according to the first embodiment of the present invention (group B1).

Trial (C).

In trial (C), the virus-like particle (VLP) formed by the vehicle fragment (group C1) and the virus-like particle (VLP) formed by the fusion peptide according to the second embodiment of the present invention (group C2) are analyzed according to the same procedure in trial (B).

Referring to FIG. 4, the horseradish peroxidase (HRP)-labelled goat anti-rabbit IgG secondary antibody can only recognize the virus-like particle (VLP) formed by the fusion peptide according to the second embodiment of the present invention (group C2).

Trial (D).

In trial (D), the virus-like particle (VLP) formed by the fusion peptide according to the first embodiment of the present invention is immobilized on the wells of a 96-well plate. The primary antibody and/or the second antibody is added into the 96-well plate according to TABLE 1. After the color reaction, the absorbance at 450 nm is measured by a spectrometer.

TABLE 1

| Group | Primary antibody | Secondary antibody |
|---|---|---|
| D1 | Rabbit anti-mouse | Horseradish |
| | IgG primary antibody | peroxidase |
| | | (HRP)-labelled goat |
| | | anti-rabbit IgG |
| | | secondary antibody |
| D2 | Mouse anti-mouse | Horseradish |
| | IgG primary antibody | peroxidase |
| | | (HRP)-labelled goat |
| | | anti-mouse IgG |
| | | secondary antibody |
| D3 | Human anti-mouse | Horseradish |
| | IgG primary antibody | peroxidase |
| | | (HRP)-labelled goat |
| | | anti-human IgG |
| | | secondary antibody |
| D4 | — | Horseradish |
| | | peroxidase |
| | | (HRP)-labelled goat |
| | | anti-rabbit IgG |
| | | secondary antibody |
| D5 | — | Horseradish |
| | | peroxidase |
| | | (HRP)-labelled goat |
| | | anti-mouse IgG |
| | | secondary antibody |
| D6 | — | Horseradish |
| | | peroxidase |
| | | (HRP)-labelled goat |
| | | anti-human IgG |
| | | secondary antibody |

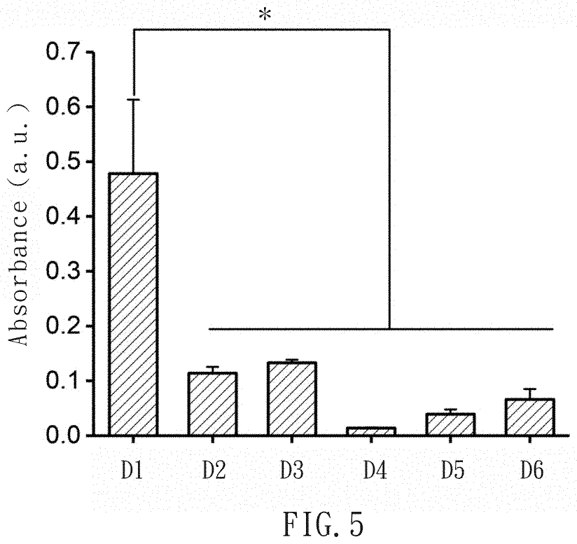

Referring to FIG. 5, the virus-like particle (VLP) formed by the fusion peptide according to the first embodiment of the present invention has a better affinity with the rabbit anti-mouse IgG primary antibody (group D1). It is worthy to note that the rabbit anti-mouse IgG primary antibody can specifically bind to the virus-like particle (VLP) formed by the fusion peptide according to the first embodiment of the present invention, indicating the capture peptide formed by the capture fragment is not enveloped by the virus-like particle (VLP) formed by the vehicle fragment.

Trial (E).

In trial (E), the mixture including the virus-like particle (VLP) and the primary antibody as shown in TABLE 2 is analyzed by agarose gel electrophoresis.

TABLE 2

| | Virus-like particle | |
|---|---|---|
| Group | (VLP) | Primary antibody |
| E1 | Virus-like particle | — |
| | (VLP) formed by the | |
| | vehicle fragment | |
| E2 | Virus-like particle | — |
| | (VLP) formed by the | |
| | fusion peptide | |
| | according to the | |
| | second embodiment | |
| | of the present | |
| | invention | |
| E3 | — | Rabbit anti-mouse |
| | | IgG primary antibody |
| | | (0.16 mg/mL) |
| E4 | Virus-like particle | Rabbit anti-mouse |
| | (VLP) formed by the | IgG primary antibody |
| | vehicle fragment | (0.16 mg/mL) |
| E5 | Virus-like particle | Rabbit anti-mouse |
| | (VLP) formed by the | IgG primary antibody |
| | fusion peptide | (0.16 mg/mL) |
| | according to the | |
| | second embodiment | |
| | of the present | |
| | invention | |
| E6 | Virus-like particle | Rabbit anti-mouse |
| | (VLP) formed by the | IgG primary antibody |
| | fusion peptide | (0.32 mg/mL) |
| | according to the | |
| | second embodiment | |
| | of the present | |
| | invention | |
| E7 | Virus-like particle | Rabbit anti-mouse |
| | (VLP) formed by the | IgG primary antibody |
| | fusion peptide | (1.60 mg/mL) |
| | according to the | |
| | second embodiment | |
| | of the present | |
| | invention | |

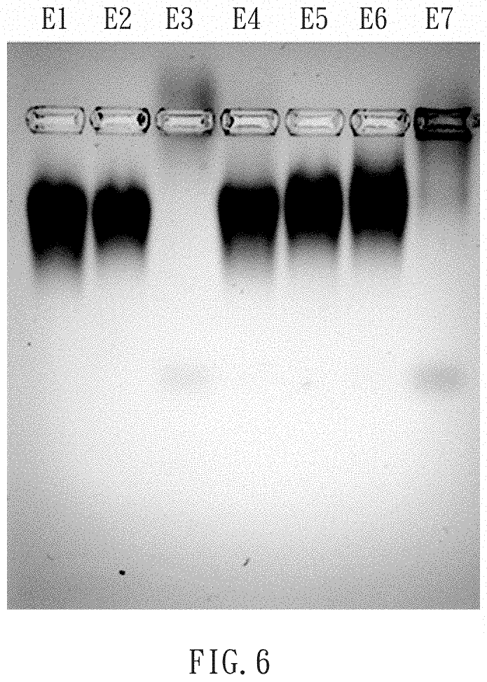

Referring to FIG. 6, the virus-like particle (VLP) formed by the fusion peptide according to the second embodiment of the present invention has a better affinity with the rabbit anti-mouse IgG primary antibody (group E7). It is worthy to note that the rabbit anti-mouse IgG primary antibody can specifically bind to the virus-like particle (VLP) formed by the fusion peptide according to the second embodiment of the present invention, indicating the capture peptide formed by the capture fragment is not enveloped by the virus-like particle (VLP) formed by the vehicle fragment.

To evaluate both the virus-like particles (VLPs) formed by the fusion peptide according to the first and second embodiments of the present invention are able to carry the anti-RBD antibody, and to evaluate both the virus-like particles (VLPs) formed by the fusion peptide according to the first and second embodiments of the present invention that carries the anti-RBD antibody are able to specifically bind to SARS-CoV-2, blocking the specific binding between SARS-CoV-2 and angiotensin-converting enzyme 2 (ACE2), the following trials are carried out.

Trial (F).

In trial (F), the platinum nanoparticle (PtNP, 70 nm, purchased from Sigma) is co-incubated with the thiolated SARS-CoV-2 spike recombinant protein (LEADGENE® SARS-CoV-2 trimeric spike protein, His tag, HEK293, CAT: 63233) at 4° C. for 12 hours to obtain the SARS-CoV-2 mimicking signal source. The SARS-CoV-2 mimicking signal source includes receptor-binding domain of SARS-CoV-2, and thus is able to specifically bind to angiotensin-converting enzyme 2 (ACE2).

Trial (G).

In trial (G), the virus-like particle (VLP) formed by the fusion peptide according to the first embodiment of the present invention is co-incubated with the anti-RBD antibody (LEADGENE® Human Anti-SARS-CoV & CoV-2 Spike Antibody (IgG)) at 37° C. for 30 minutes. The capture peptide which is formed by the capture fragment of the fusion peptide and is not enveloped by the virus-like particle (VLP) can bind to the Fc domain of the anti-RBD antibody to form the virus-like particle (VLP) formed by the fusion peptide according to the first embodiment of the present invention carrying the anti-RBD antibody.

Moreover, the virus-like particle (VLP) formed by the fusion peptide according to the second embodiment of the present invention is co-incubated with the anti-RBD antibody (LEADGENE® Human Anti-SARS-CoV & CoV-2 Spike Antibody (IgG)) at 37° C. for 30 minutes. The capture peptide which is formed by the capture fragment of the fusion peptide and is not enveloped by the virus-like particle (VLP) can bind to the Fc domain of the anti-RBD antibody to form the virus-like particle (VLP) formed by the fusion peptide according to the second embodiment of the present invention carrying the anti-RBD antibody.

Trial (H).

In trial (H), angiotensin-converting enzyme 2 (ACE2) is diluted to a solution of 200 ng/mL in a carbonate-bicarbonate buffer (CBC buffer, 0.05 M Na₂CO₃ and 0.05 M NaHCO₃, pH 9.6). The diluted angiotensin-converting enzyme 2 (ACE2) solution (100 μL) is added into each well of a 96-well plate. After incubation overnight at 4° C., the wells are washed with the tris(hydroxymethyl)aminomethane (Tris) buffer (TBST) with polysorbate 20 (Tween 20) for three times, then blocked with the blocking buffer (5% bovine serum albumin (BSA), dissolved in TBST) at room temperature for 1 hour. Each well is then washed with the tris(hydroxymethyl)aminomethane (Tris) buffer (TBST) with polysorbate 20 (Tween 20) for three time to obtain the well immobilized with angiotensin-converting enzyme 2 (ACE2).

The mixture (100 μL) as shown in TABLE 3 is added to each well, incubating at room temperature for 1 hour. Each well is washed with the tris(hydroxymethyl)aminomethane (Tris) buffer (TBST) with polysorbate 20 (Tween 20) for three time. Next, horseradish peroxidase (HRP)-labelled angiotensin-converting enzyme 2 (ACE2) antibody is used to stain each well. Finally, the commercial chromogenic solution (with 3,3′,5,′-tetramethylbenzidine (TMB) and hydrogen peroxide (H₂O₂), 100 μL) is added to each well, and the reaction is terminated by adding the aqueous hydrochloric acid (HCl) solution (1 M). The absorbance of each well at 450 nm is measured by the spectrometer (SpectraMax M2).

TABLE 3

| | SARS-CoV-2 | |
|---|---|---|
| | mimicking signal | |
| Group | source¹ | Anti-RBD antibody |
| H1 | + | — |
| H2 | + | Anti-RBD antibody² |
| H3 | + | Virus-like particle |
| | | (VLP) formed by the |
| | | fusion peptide |
| | | according to the first |
| | | embodiment of the |
| | | present invention |
| | | carrying the anti-RBD |
| | | antibody³ |

¹with 2.5 ng/mL of receptor-binding domain of SARS-CoV-2, dissolved in phosphate buffeted saline (PBS);
²with 10 ng/mL of anti-RBD antibody, dissolved in phosphate buffeted saline (PBS);
³with 10 ng/mL of anti-RBD antibody, dissolved in phosphate buffeted saline (PBS).

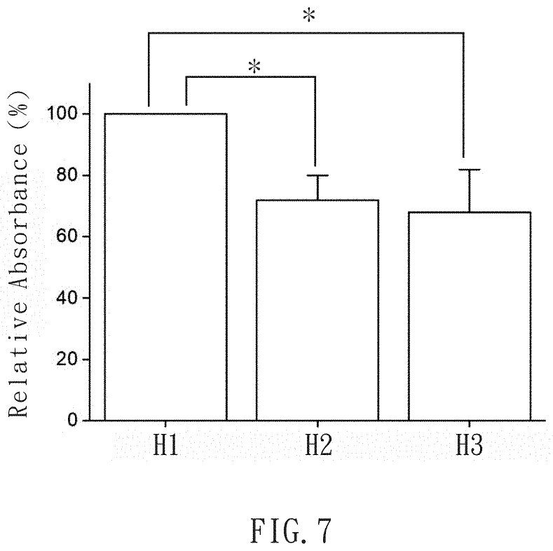

Referring to FIG. 7, both the anti-RBD antibody and the virus-like particle (VLP) formed by the fusion peptide according to the first embodiment of the present invention carrying the anti-RBD antibody can specifically bind to the SARS-CoV-2 mimicking signal source, preventing angiotensin-converting enzyme 2 (ACE2) immobilized on the well from binding to the SARS-CoV-2 mimicking signal source (groups H2 and H3).

Trial (I).

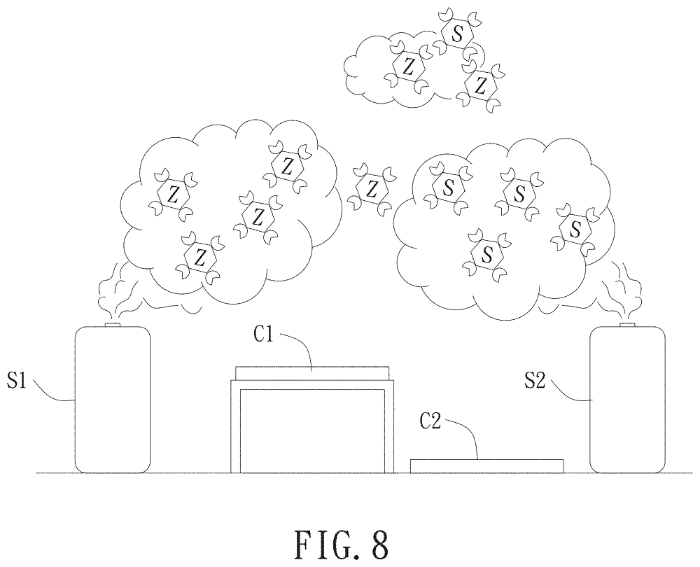

Referring to FIG. 8, in trial (I), a first sprayer S1 and a second sprayer S2 are set in a sealed glass box with a length of 60 cm, a width of 45 cm and a height of 30 cm. An upper chip C1 and a lower chip C2 are set between the first sprayer S1 and the second sprayer S2. The upper chip C1 is set onto a frame with a height of 10 cm, and thus is positioned with a higher position compared to the lower chip C2. Both the upper chip C1 and the lower chip C2 are immobilized with angiotensin-converting enzyme 2 (ACE2) on its surface.

The first sprayer S1 and the second sprayer S2 are filled in the solutions shown in TABLE 4 and are turned on to form ultrasonic spray of respective solutions. After spraying for 60 minutes, the first sprayer S1 and the second sprayer S2 are turned off, and the ultrasonic spray of respective solutions is settled for 60 minutes. After washing with the tris(hydroxymethyl)aminomethane (Tris) buffer (TBST) with polysorbate 20 (Tween 20) for three time, the upper chip C1 and the lower chip C2 are stained by the horseradish peroxidase (HRP)-labelled angiotensin-converting enzyme 2 (ACE2) antibody. Finally, the commercial chromogenic solution (with 3,3′,5,′-tetramethylbenzidine (TMB) and hydrogen peroxide (H₂O₂), 100 μL) is added to the upper chip C1 and the lower chip C2, and the reaction is terminated by adding the aqueous hydrochloric acid (HCl) solution (1 M). The absorbance of the upper chip C1 and the lower chip C2 at 450 nm is measured by the spectrometer (SpectraMax M2).

TABLE 4

| Group | First sprayer S1 | Second sprayer S2 |
|---|---|---|
| I1 | Phosphate buffered | SARS-CoV-2 |
| | saline (PBS) | mimicking signal |
| | | source² |
| I2 | Virus-like particle | SARS-CoV-2 |
| | (VLP) formed by the | mimicking signal |
| | fusion peptide | source² |
| | according to the | |
| | second embodiment | |
| | of the present | |
| | invention carrying the | |
| | anti-RBD antibody¹ | |

¹with 10 ng/mL of anti-RBD antibody, dissolved in phosphate buffeted saline (PBS);
²with 2.5 ng/mL of SARS-CoV-2 [symbol] receptor-binding domain, dissolved in phosphate buffeted saline (PBS).

Referring to FIG. 9, compared to the upper chip C1 and the lower chip C2 in group IL the upper chip C1 and the lower chip C2 in group 12 has a lower absorbance, respectively. Moreover, the lower chip C2 is positioned with a lower position compared to the higher chip C1; and thus, the virus-like particle (VLP) formed by the fusion peptide according to the second embodiment of the present invention carrying the anti-RBD antibody that is sprayed by the first sprayer S1 has enough time to specifically bind to the SARS-CoV-2 mimicking signal source that is sprayed by the second sprayer S2. As a result, the lower chip C2 has a lower absorbance than the higher chip C1 in group 12.

Trial (J).

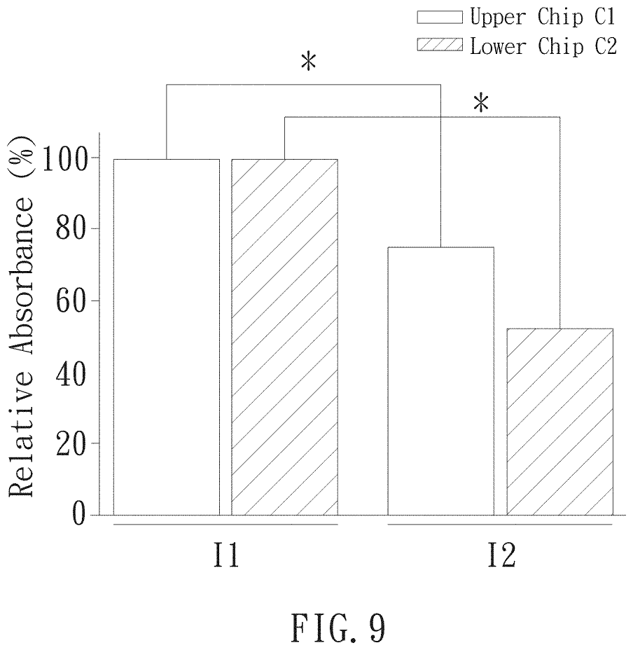

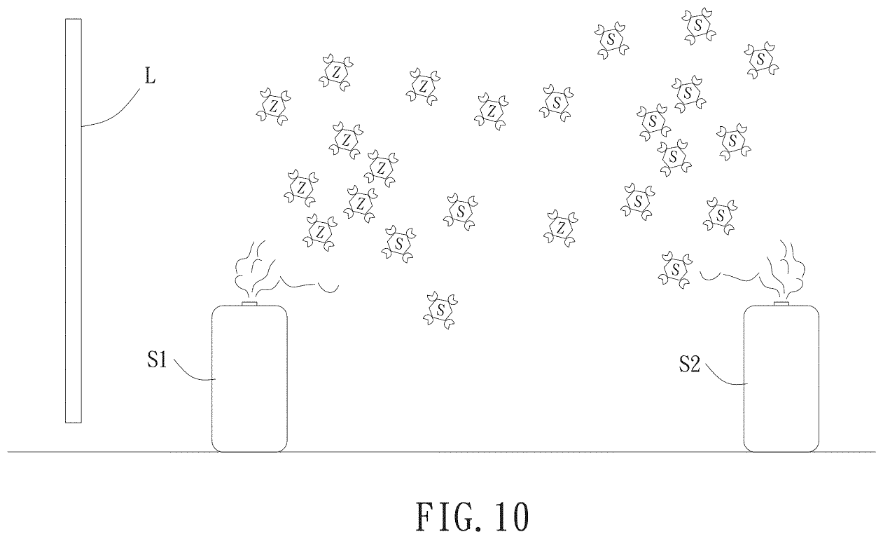

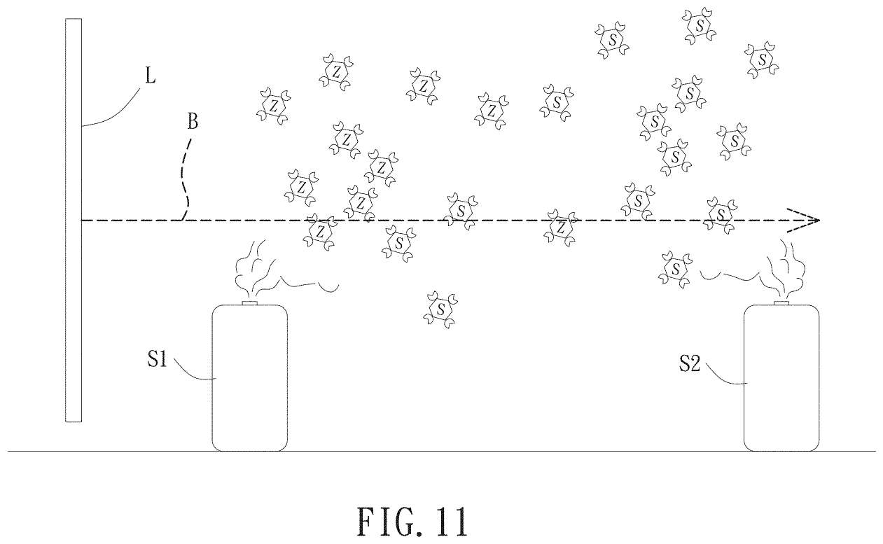

Referring to FIG. 10, in trial (J), the first sprayer S1 and the second sprayer S2 are set in the sealed glass box with a length of 60 cm, a width of 45 cm and a height of 30 cm. The first sprayer S1 and the second sprayer S2 are filled in the solutions shown in TABLE 5 and are turned on to form ultrasonic spray of respective solutions. After spraying for 5 minutes, the first sprayer S1 and the second sprayer S2 are turned off, and the ultrasonic spray of respective solutions is settled for 60 minutes. After that, referring to FIG. 11, a light source L is turned on to produce a blue laser beam B (50 mW, with a wavelength of 405 nm) passing through the sealed glass box. The optical path formed by the blue laser beam B at 0 minute (that is, the time point that the light source L is turned on) and 40 minutes are photographed.

TABLE 5

| Group | First sprayer S1 | Second sprayer S2 |
|---|---|---|
| J1 | Phosphate buffered | SARS-CoV-2 |
| | saline (PBS) | mimicking signal |
| | | source² |
| J2 | Virus-like particle | SARS-CoV-2 |
| | (VLP) formed by the | mimicking signal |
| | fusion peptide | source² |
| | according to the | |
| | second embodiment | |
| | of the present | |
| | invention carrying the | |
| | anti-RBD antibody¹ | |

¹with 10 ng/mL of anti-RBD antibody, dissolved in phosphate buffeted saline (PBS);
²with 2.5 ng/mL of receptor-binding domain of SARS-CoV-2, dissolved in phosphate buffeted saline (PBS).

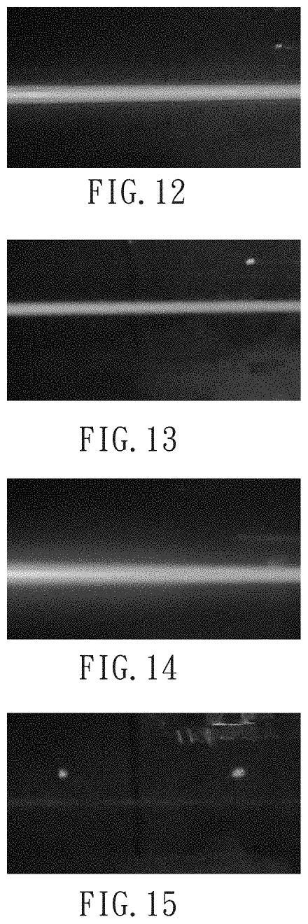

Referring to FIGS. 12 and 13, in the situation that only the SARS-CoV-2 mimicking signal source is suspended in the sealed glass box, after turning on the light source L that emitting the blue laser beam B for 40 minutes, the optical path formed by the blue laser beam B can be observed, indicating that the SARS-CoV-2 mimicking signal source is still suspended in the air. Moreover, referring to FIGS. 14 and 15, in the situation that both the SARS-CoV-2 mimicking signal source and the virus-like particle (VLP) formed by the fusion peptide according to the second embodiment of the present invention carrying the anti-RBD antibody are suspended in the sealed glass box, after turning on the light source L that emitting the blue laser beam B for 40 minutes, the optical path formed by the blue laser beam B cannot be observed due to the SARS-CoV-2 mimicking signal source is quickly settled with the virus-like particle (VLP) formed by the fusion peptide according to the second embodiment of the present invention carrying the anti-RBD antibody.

Accordingly, instead of enveloping in the virus-like particle formed by the vehicle fragment, the fusion peptide for forming virus-like particle according to the present invention has the capture peptide formed by the capture fragment that is exposed outside the virus-like particle. Therefore, the capture peptide can specifically bind to a target biomolecule to form the virus-like particle with the target biomolecule, and the target biomolecule can be delivered to a predetermined location by the virus-like particle. Alternatively, by the specific binding between the capture peptide and the target biomolecule, the targeting of the virus-like particle can also be improved.

Moreover, the virus-like particle carrying the target biomolecule is formed by a reversible specific binding (such as the interaction of hydrogen bond, van der Waals force, electrostatic force, hydrophobic interaction, etc.) between the capture peptide and the target biomolecule. Therefore, the process for conjugating the target biomolecule to the virus-like particle (VLP) using the crosslinker can be omitted. As such, the virus-like particle (VLP) carrying the target biomolecule can be manufactured at a decreased cost, and the virus-like particle (VLP) carrying the target biomolecule can be manufactured with a better yield.

Also, the capture peptide with an IgG Fc-binding domain that is able to bind to a Fc domain of an antibody (such as the Fc domain of an anti-RBD antibody); and thus, the fusion peptide can form the virus-like particle (VLP) carrying the antibody by the specific binding between the IgG Fc-binding domain and the Fc domain. The antibody carried by the virus-like particle (VLP) can specifically bind to a virus particle (such as such as specifically bind to the virus particle of SARS-CoV-2 via the receptor-binding domain of SARS-CoV-2). As a result, the specific binding between the virus particle and the receptor of the target cell (such as angiotensin-converting enzyme 2 (ACE2)) can be blocked, and the infection of the target cell by the virus particle can be prevented.

In addition, the capture peptide with a z domain that is able to bind to a Fc domain of an antibody (such as the Fc domain of an anti-RBD antibody); and thus, the fusion peptide can form the virus-like particle (VLP) carrying the antibody by the specific binding between the z domain and the Fc domain. The antibody carried by the virus-like particle (VLP) can specifically bind to a virus particle (such as such as specifically bind to the virus particle of SARS-CoV-2 via the receptor-binding domain of SARS-CoV-2). As a result, the specific binding between the virus particle and the receptor of the target cell (such as angiotensin-converting enzyme 2 (ACE2)) can be blocked, and the infection of the target cell by the virus particle can be prevented.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A fusion peptide configured to form a virus-like particle, comprising:
a vehicle fragment configured to form a virus-like particle; and
a capture fragment connected to the vehicle fragment, wherein the capture fragment is configured to form a capture peptide, wherein the capture peptide is not enveloped by the virus-like particle, and wherein the capture peptide is configured to specific bind to a target biomolecule.

2. The fusion peptide configured to form the virus-like particle as claimed in claim 1, further comprising a linker fragment connected between the vehicle fragment and the capture fragment.

3. The fusion peptide configured to form the virus-like particle as claimed in claim 1, wherein the vehicle fragment has an amino acid sequence set forth as SEQ ID NO: 3, and wherein the capture fragment has an amino acid sequence set forth as SEQ ID NO: 4.

4. The fusion peptide configured to form the virus-like particle as claimed in claim 3, wherein the fusion peptide consists of an amino acid sequence set forth as SEQ ID NO: 6.

5. The fusion peptide configured to form the virus-like particle as claimed in claim 1, wherein the vehicle fragment has an amino acid sequence set forth as SEQ ID NO: 3, and wherein the capture fragment has an amino acid sequence set forth as SEQ ID NO: 8.

6. The fusion peptide configured to form the virus-like particle as claimed in claim 5, wherein the fusion peptide consists of an amino acid sequence set forth as SEQ ID NO: 10.
